(19) United States
(12) Patent Application Publication
(10) Pub. No.: US 2007/0014791 A1
(43) Pub. Date: Jan. 18, 2007

(54) Rage-related methods and copositions for treating glomerular injury

(76) Inventors: Ann Marie Schmidt, Franklin Lakes, NJ (US); Vivette D'Agati, Tenafly, NJ (US)

Correspondence Address: COOPER & DUNHAM, LLP, 1185 AVENUE OF THE AMERICAS, NEW YORK, NY, 10036, US

(21) Appl. No.: 10/570,674

(22) PCT Filed: Sep. 3, 2004
(86) PCT No.: PCT/US04/28712

§ 371 Date: Jun. 27, 2006

Related U.S. Application Data
(60) Provisional application No. 60/500,663, filed on Sep. 5, 2003.

(51) Int. Cl. A61K 39/395; A61K 31/704

(52) U.S. Cl. 424/143.1; 514/34

(57) ABSTRACT

This invention provides methods, compositions and articles of manufacture for inhibiting the onset of and treating glomerular injury. The instant invention is based on the blockade of RAGE and/or RAGE G82S function.

Throughout this application, various publications are referenced. Full citations for these publications may be found immediately preceding the claims. The disclosures of these publications are hereby incorporated by reference into this application in order to more fully describe the state of the art as of the date of the invention described and claimed herein.

### BACKGROUND OF THE INVENTION

Primary or secondary focal segmental glomerulosclerosis (FSGS) encompasses a range of diseases characterized by glomerular and tubulointerstitial fibrosis that often progress, unhaltingly, to irreversible renal scarring and failure in human subjects (1). Secondary cases of FSGS may emerge in the face of chronic disease (hemodynamic, immunologic or metabolic). However, in both cases of primary and secondary disease, despite many years of study, there is no definitive understanding of the molecular mechanisms that underlie these disorders. As such, insights into means to prevent/treat these disorders have not been elucidated.

Key steps in identifying rational therapeutic targets for these diseases, however, may emerge from animal studies. Development of FSGS by agents that incite pathways linked to glomerular fibrosis and hyperpermeability are useful as a means to track the early, initiating events and the later, amplified consequences of proteinuria and renal scarring. In this context, multiple studies have employed administration of agents such as puromycin or adriamycin (ADR) to rats, to induce processes analogous to human FSGS in the kidney (2-4). In addition, other studies in rats have included the induction of Passive Heymann Nephritis as a means to induce irreversible glomerular injury (5). Overall, these studies in rats have been frustrated by the inability to precisely link activation of specific cells to the pathogenesis and/or progression of GS upon disease induction.

A paucity of mouse models existed for the study of FSGS-like diseases until the first description of ADR-induced toxicity in mice (6-7). In 2000, Wang and colleagues reported on the impact of ADR up to 42 days (6 weeks) after administration of ADR (9). Male BALB/c mice, 20 to 25 gm, were injected with ADR, 10.5 mg/kg, by IV injection. These investigators carefully followed the time course of events in the ADR-treated mice and observed the following (9).

First, overt proteinuria developed in all mice by day 5. Proteinuria persisted throughout 6 weeks of study. Only 35.7% of mice developed hematuria but 53.6% developed leukocyturia.

Second, levels of serum albumin were consistently lower in ADR-treated mice vs controls beginning one week after ADR treatment.

Third, creatinine clearance declined with time and was significantly decreased compared to control mice 4 weeks post-ADR.

Fourth, by week 6, tubular atrophy and intratubular cast formation with tubulointerstitial expansion had occurred and was widely seen in the cortex. Extensive FGS and severe interstitial fibrosis and inflammation were present. Global sclerosis was observed in many glomeruli.

Fifth, by EM, effacement of foot processes of podocytes had occurred. At week 1, effacement was segmental, but global by week 6. Control mice failed to demonstrate any epithelial cell abnormalities at any point.

Importantly, in this study, cellular infiltration and inflammation were examined.

Sixth, at early and later times after ADR, CD4+ and CD8+ T cells, and macrophages were significantly increased in the kidneys of ADR-treated mice. These cell types were found both in the interstitium as well as in the glomeruli after injury. Infiltration of inflammatory cells was noted quite early after ADR, within the first 24 hours, and persisted for up to weeks after ADR. These findings support the premise that inflammation, at least in part, contributes as an early trigger, and/or late progression factor in the molecular pathways leading to sustained glomerular perturbation, fibrosis and albuminuria that converge to cause renal dysfunction.

These studies highlighted that even 6 weeks after ADR, progressive renal injury, proteinuria and decreased creatinine clearance were features of the disease. In addition, new insights into proinflammatory mechanisms into the disease process were uncovered by time course examination of cellular infiltration after ADR. Other studies have, in fact, confirmed inflammatory cell infiltration into the ADR-treated kidney (9). Indeed, the observation that human FSGS is typified by differentiation of podocytes into MP-like cells, along with inflammatory cell infiltration from the periphery (MP and T lymphocytes) in the interstitium, periglomerular regions and glomeruli (1, 10-12) is compatible with the concept put forth in the ADR-induced murine model of FSGS, that is, it is plausible that inflammatory stimuli importantly contribute to the pathogenesis and/or progression of FSGS.

In parallel with progressive renal dysfunction and scarring in primary or secondary FSGS syndromes in human subjects (and murine models), injury and depletion of glomerular podocytes, eventuating in podocyte “insufficiency” and capillary collapse, have been implicated as important steps in the development of FSGS (13, 14). In most cases of nephrotic syndrome, podocyte foot process effacement is considered an early manifestation of injury, and is followed by a continuum of progressive podocyte damage characterized by vacuolization, pseudocyst formation, detachment of podocytes from the GBM; processes that lead to irreversible loss/apoptosis of podocytes (15).

Key evidence that podocytes are not mere bystanders, but rather active participants in molecular pathways of injury, was highlighted by recent studies in TGF-β overexpressing transgenic mice. In those mice, marked upregulation of Smad 7 was observed in damaged podocytes. Both TGF-β and Smad7 were associated with apoptosis in cultured podocytes. In the former case, activation of p38 MAP kinase and caspase-3 were key intermediary steps in TGF-β-induced apoptosis. In the latter case, suppressed nuclear translocation of the cell survival factor NF-kB led to Smad7-induced podocyte apoptosis (16). These studies highlight the concept that activation of cell signalling and modulation of gene expression in the podocyte may be early events in the development of FSGS, and thus, may contribute to the pathogenesis of this disease.

It is important to note that the concept of key roles for podocytes in the pathogenesis/progression of glomerular dysfunction have parallels in diabetes. Diabetes is a highly complex environment in which multiple contributing pathways, such as accumulation/activation of Advanced Glycation Endproducts, activation of PKC, especially the β isoform, as well as hyperglycemia itself are implicated in the pathogenesis of this disorder (17-19). Evidence is accumulating that podocytes are perturbed early in diabetes, and that their products, such as VEGF, may contribute to cellular dysfunction in this disorder (20-25). As in FSGS and FSGS-like disorders, the case for the podocyte as bystander vs contributory agent to the pathogenesis and progression of glomerular injury remains to be rigorously tested.

Although inhibiting RAGE has been implicated in treating symptoms of diabetes (35), the literature does not provide a basis for concluding that inhibiting the binding of RAGE to its ligands could play a role in treating or preventing glomerular injury.

### SUMMARY OF THE INVENTION

This invention provides a method for inhibiting the onset of a glomerular injury in a subject comprising administering to the subject a prophylactically effective amount of an agent that inhibits binding between RAGE and/or RAGE G82S and a ligand thereof.

This invention further provides a method for treating a glomerular injury in a subject comprising administering to the subject a therapeutically effective amount of an agent that inhibits binding between RAGE and/or RAGE G82S and a ligand thereof.

This invention further provides a method for inhibiting the onset of glomerulosclerosis, proteinuria or albunuria in a subject comprising administering to the subject a prophylactically effective amount of an agent that inhibits binding between RAGE and/or RAGE G82S and a ligand thereof.

This invention further provides a method for treating glomerulosclerosis, proteinuria or albunuria in a subject comprising administering to the subject a therapeutically effective amount of an agent that inhibits binding between RAGE and/or RAGE G82S and a ligand thereof.

This invention further provides an article of manufacture comprising a packaging material having therein an agent that inhibits binding between RAGE and/or RAGE G82S and a ligand thereof, wherein the packaging material has affixed thereto a label indicating a use for the agent for inhibiting the onset of glomerular injury in a subject.

This invention further provides an article of manufacture comprising a packaging material having therein an agent that inhibits binding between RAGE and/or RAGE G82S and a ligand thereof, wherein the packaging material has affixed thereto a label indicating a use for the agent for inhibiting the onset of glomerulosclerosis, proteinuria or albuminuria in a subject.

This invention further provides an article of manufacture comprising a packaging material having therein an agent that inhibits binding between RAGE and/or RAGE G82S and a ligand thereof, wherein the packaging material has affixed thereto a label indicating a use for the agent for treating a glomerular injury in a subject.

Finally, this invention provides an article of manufacture comprising a packaging material having therein an agent that inhibits binding between RAGE and/or RAGE G82S and a ligand thereof, wherein the packaging material has affixed thereto a label indicating a use for the agent for treating glomerulosclerosis, proteinuria or albuminuria in a subject.

### BRIEF DESCRIPTION OF THE FIGURES

FIG. 1. Administration of ADR to BALB/C mice: effects of sRAGE. BALB/c mice were treated with ADR or control (saline). ADR-treated mice received sRAGE or PBS. At 2 weeks after ADR, kidney wt/body wt ratio and mesangial area & mesangial/glomerular fraction determined. N=5 mice/group. Statistical considerations are indicated in the figures.

FIG. 2. Administration of ADR to BALB/c mice: effects of sRAGE. BALB/c mice were treated with ADR or control (saline). ADR-treated mice received sRAGE or PBS. At 6 weeks after ADR, kidney wt/body wt ratio and mesangial area & mesangial/glomerular fraction determined. N=5 mice/group. Statistical considerations are indicated in the figures.

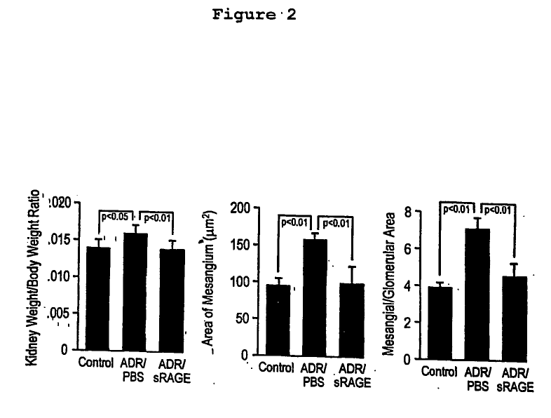

FIG. 3. Blockade of RAGE suppresses albuminuria after administration of ADR. At 2 and 6 weeks after ADR, urine albumin/creatinine ratio was determined. N=5 mice/condition. N=5 mice/condition. Statistical considerations are indicated in the figure.

### DETAILED DESCRIPTION OF THE INVENTION

Definitions

“Agent” shall include, without limitation, an organic compound, a nucleic acid, a polypeptide, a lipid, and a carbohydrate. Agents include, for example, agents which are known with respect to structure and/or function, and those which are not known with respect to structure or function.

“Antibody” shall include, by way of example, both naturally occurring and non-naturally occurring antibodies. Specifically, this term includes polyclonal and monoclonal antibodies, and antigen-binding fragments thereof. Furthermore, this term includes chimeric antibodies and wholly synthetic antibodies, and antigen-binding fragments thereof.

As used herein, “inhibit,” when used in connection with the binding between RAGE and/or RAGE G82S with a ligand thereof, shall mean to reduce such binding. In one embodiment, “inhibit” shall mean to eliminate such binding.

“Inhibiting” the onset of a disorder shall mean either lessening the likelihood of the disorder's onset, or preventing the onset of the disorder entirely. In the preferred embodiment, inhibiting the onset of a disorder means preventing its onset entirely.

“Subject” shall mean any animal, such as a human, non-human primate, mouse, rat, guinea pig or rabbit.

“Treating” a disorder shall mean slowing, stopping or reversing the disorder's progression. In the preferred embodiment, treating a disorder means reversing the disorder's progression, ideally to the point of eliminating the disorder itself. As used herein, ameliorating a disorder and treating a disorder are equivalent.

Embodiments of the Invention

This invention provides methods for inhibiting the onset of and treating glomerular injury. This invention is based on the surprising discovery of a correlation between suppressing glomerular injury in a non-diabetic subject and blocking RAGE and/or RAGE G82S function.

Specifically, this invention provides a method for inhibiting the onset of a glomerular injury in a subject comprising administering to the subject a prophylactically effective amount of an agent that inhibits binding between RAGE and/or RAGE G82S and a ligand thereof.

In one embodiment of the instant method, the glomerular injury is associated with reduced removal of toxins. In another embodiment, the glomerular injury is associated with glomerulosclerosis. In a further embodiment, the glomerular injury is associated with proteinuria. In yet a further embodiment, the glomerular injury is associated with albuminuria.

In the preferred embodiment of the instant method, the subject is human. In one embodiment the subject is afflicted with diabetes. In another embodiment of the instant method, the subject has been afflicted with diabetes for less than 20 years. In a further embodiment, the subject is not afflicted with diabetes. In yet a further embodiment, the subject is receiving or is about to receive a chemotherapy drug. In yet a further embodiment, the chemotherapy drug is adriamycin. In yet a further embodiment, the chemotherapy drug is selected from the following: 5-fluorouracil; Actinomycin D; Alpha interferon; Bleomycin; Cisplatin; Cyclophosphamide; Dexamethasone; Doxorubicin; Epoetin alfa; Etoposide; Gleevec; Herceptin; Interferon alfa; Interleukin-2; Interleukin-11; Methotrexate; Neupogen; Nitrogen Mustard; Paclitaxel; Prednisolone; Prednisone; PROCRIT; Rituximab; Tamoxifen; Thalidomide; Vinblastine; and Vincristine. Additional chemotherapy drugs are envisioned, and are listed in chemocare.com (http://www.chemocare.com/bio/default.sps).

In one embodiment of the instant method, the agent is soluble RAGE. In another embodiment, the agent is soluble RAGE G82S. In a further embodiment, the agent is an antibody directed to RAGE. In yet a further embodiment, the agent is an antibody directed to RAGE G82S.

This invention further provides a method for treating a glomerular injury in a subject comprising administering to the subject a therapeutically effective amount of an agent that inhibits binding between RAGE and/or RAGE G82S and a ligand thereof.

In one embodiment of the instant method, the glomerular injury is associated with reduced removal of toxins. In another embodiment, the glomerular injury is associated with glomerulosclerosis. In a further embodiment, the glomerular injury is associated with proteinuria. In yet a further embodiment, the glomerular injury is associated with albuminuria.

In the preferred embodiment of the instant method, the subject is human. In one embodiment, the subject is not afflicted with diabetes. In another embodiment, the subject is receiving or is about to receive a chemotherapy drug. In a further embodiment, the chemotherapy drug is adriamycin. In yet a further embodiment, the chemotherapy drug is selected from the following: 5-fluorouracil; Actinomycin D; Alpha interferon; Bleomycin; Cisplatin; Cyclophosphamide; Dexamethasone; Doxorubicin; Epoetin alfa; Etoposide; Gleevec; Herceptin; Interferon alfa; Interleukin-2; Interleukin-11; Methotrexate; Neupogen; Nitrogen Mustard; Paclitaxel; Prednisolone; Prednisone; PROCRIT; Rituximab; Tamoxifen; Thalidomide; Vinblastine; and Vincristine. Additional chemotherapy drugs are envisioned, and are listed in chemocare.com (http://www.chemocare.com/bio/default.sps).

In one embodiment of the instant method, the agent is soluble RAGE. In another embodiment, the agent is soluble RAGE G82S. In a further embodiment, the agent is an antibody directed to RAGE. In yet a further embodiment, the agent is an antibody directed to RAGE G82S.

This invention further provides a method for inhibiting the onset of glomerulosclerosis, proteinuria or albunuria in a subject comprising administering to the subject a prophylactically effective amount of an agent that inhibits binding between RAGE and/or RAGE G82S and a ligand thereof.

In the preferred embodiment of the instant method, the subject is human. In one embodiment the subject is afflicted with diabetes. In another embodiment of the instant method, the subject has been afflicted with diabetes for less than 20 years. In a further embodiment, the subject is not afflicted with diabetes. In yet a further embodiment, the subject is receiving or is about to receive a chemotherapy drug. In yet a further embodiment, the chemotherapy drug is adriamycin. In yet a further embodiment, the chemotherapy drug is selected from the following: 5-fluorouracil; Actinomycin D; Alpha interferon; Bleomycin; Cisplatin; Cyclophosphamide; Dexamethasone; Doxorubicin; Epoetin alfa; Etoposide; Gleevec; Herceptin; Interferon alfa; Interleukin-2; Interleukin-11; Methotrexate; Neupogen; Nitrogen Mustard; Paclitaxel; Prednisolone; Prednisone; PROCRIT; Rituximab; Tamoxifen; Thalidomide; Vinblastine; and Vincristine. Additional chemotherapy drugs are envisioned, and are listed in chemocare.com (http://www.chemocare.com/bio/default.sps).

In one embodiment of the instant method, the agent is soluble RAGE. In another embodiment, the agent is soluble RAGE G82S. In a further embodiment, the agent is an antibody directed to RAGE. In yet a further embodiment, the agent is an antibody directed to RAGE G82S.

This invention further provides a method for treating glomerulosclerosis, proteinuria or albunuria in a subject comprising administering to the subject a therapeutically effective amount of an agent that inhibits binding between RAGE and/or RAGE G82S and a ligand thereof.

In the preferred embodiment of the instant method, the subject is human. In one embodiment, the subject is not afflicted with diabetes. In another embodiment, the subject is receiving or is about to receive a chemotherapy drug. In a further embodiment, the chemotherapy drug is adriamycin. In yet a further embodiment, the chemotherapy drug is selected from the following: 5-fluorouracil; Actinomycin D; Alpha interferon; Bleomycin; Cisplatin; Cyclophosphamide; Dexamethasone; Doxorubicin; Epoetin alfa; Etoposide; Gleevec; Herceptin; Interferon alfa; Interleukin-2; Interleukin-11; Methotrexate; Neupogen; Nitrogen Mustard; Paclitaxel; Prednisolone; Prednisone; PROCRIT; Rituximab; Tamoxifen; Thalidomide; Vinblastine; and Vincristine. Additional chemotherapy drugs are envisioned, and are listed in chemocare.com (http://www.chemocare.com/bio/default.sps).

In one embodiment of the instant method, the agent is soluble RAGE. In another embodiment, the agent is soluble RAGE G82S. In a further embodiment, the agent is an antibody directed to RAGE. In yet a further embodiment, the agent is an antibody directed to RAGE G82S.

Determining a therapeutically or prophylactically effective amount of agent can be done based on animal data using routine computational methods. In one embodiment, the therapeutically or prophylactically effective amount contains between about 1 ng and about Ig of protein, as applicable. In another embodiment, the effective amount contains between about long and about 100 mg of protein, as applicable. In a further embodiment, the effective amount contains between about 100 ng and about 10 mg of the protein, as applicable. In a yet a further embodiment, the effective amount contains between about 1 μg and about 1 mg of the protein, as applicable. In a yet a further embodiment, the effective amount contains between about 10 μg and about 100 μg of the protein, as applicable. In a yet a further embodiment, the effective amount contains between about 100 μg and about 10 mg of the protein, as applicable. In yet a further embodiment, the effective amount of agent, wherein the agent is soluble RAGE, is administered to the subject at a rate from about 2 μg/kg/hr to about 100 μg/kg/hr (e.g. about 5, 10, 25, 50 or 75 μg/kg/hr).

In this invention, administering agents can be effected or performed using any of the various methods and delivery systems known to those skilled in the art. The administering can be performed, for example, intravenously, orally, via implant, transmucosally, transdermally, intramuscularly, and subcutaneously. The following delivery systems, which employ a number of routinely used pharmaceutical carriers, are only representative of the many embodiments envisioned for administering the instant compositions.

Injectable drug delivery systems include solutions, suspensions, gels, microspheres and polymeric injectables, and can comprise excipients such as solubility-altering agents (e.g., ethanol, propylene glycol and sucrose) and polymers (e.g., polycaprylactones and PLGA's). Implantable systems include rods and discs, and can contain excipients such as PLGA and polycaprylactone.

Oral delivery systems include tablets and capsules. These can contain excipients such as binders (e.g., hydroxypropylmethylcellulose, polyvinyl pyrilodone, other cellulosic materials and starch), diluents (e.g., lactose and other sugars, starch, dicalcium phosphate and cellulosic materials), disintegrating agents (e.g., starch polymers and cellulosic materials) and lubricating agents (e.g., stearates and talc).

Transmucosal delivery systems include patches, tablets, suppositories, pessaries, gels and creams, and can contain excipients such as solubilizers and enhancers (e.g., propylene glycol, bile salts and amino acids), and other vehicles (e.g., polyethylene glycol, fatty acid esters and derivatives, and hydrophilic polymers such as hydroxypropylmethylcellulose and hyaluronic acid).

Dermal delivery systems include, for example, aqueous and nonaqueous gels, creams, multiple emulsions, microemulsions, liposomes, ointments, aqueous and nonaqueous solutions, lotions, aerosols, hydrocarbon bases and powders, and can contain excipients such as solubilizers, permeation enhancers (e.g., fatty acids, fatty acid esters, fatty alcohols and amino acids), and hydrophilic polymers (e.g., polycarbophil and polyvinylpyrolidone). In one embodiment, the pharmaceutically acceptable carrier is a liposome or a transdermal enhancer.

Solutions, suspensions and powders for reconstitutable delivery systems include vehicles such as suspending agents (e.g., gums, zanthans, cellulosics and sugars), humectants (e.g., sorbitol), solubilizers (e.g., ethanol, water, PEG and propylene glycol), surfactants (e.g., sodium lauryl sulfate, Spans, Tweens, and cetyl pyridine), preservatives and antioxidants (e.g., parabens, vitamins E and C, and ascorbic acid), anti-caking agents, coating agents, and chelating agents (e.g., EDTA).

In one embodiment of this invention, the delivery system used comprises more than water alone, or more than buffer alone.

This invention further provides an article of manufacture comprising a packaging material having therein an agent that inhibits binding between RAGE and/or RAGE G82S and a ligand thereof, wherein the packaging material has affixed thereto a label indicating a use for the agent for inhibiting the onset of glomerular injury in a subject. This invention further provides an article of manufacture comprising a packaging material having therein an agent that inhibits binding between RAGE and/or RAGE G82S and a ligand thereof, wherein the packaging material has affixed thereto a label indicating a use for the agent for inhibiting the onset of glomerulosclerosis, proteinuria or albuminuria in a subject.

In the preferred embodiment of the instant articles of manufacture, the subject is human. In one embodiment the subject is afflicted with diabetes. In another embodiment of the instant methods, the subject has been afflicted with diabetes for less than 20 years. In a further embodiment, the subject is not afflicted with diabetes. In yet a further embodiment, the subject is receiving or is about to receive a chemotherapy drug. In yet a further embodiment, the chemotherapy drug is adriamycin. In yet a further embodiment, the chemotherapy drug is selected from the following: 5-fluorouracil; Actinomycin D; Alpha interferon; Bleomycin; Cisplatin; Cyclophosphamide; Dexamethasone; Doxorubicin; Epoetin alfa; Etoposide; Gleevec; Herceptin; Interferon alfa; Interleukin-2; Interleukin-11; Methotrexate; Neupogen; Nitrogen Mustard; Paclitaxel; Prednisolone; Prednisone; PROCRIT; Rituximab; Tamoxifen; Thalidomide; Vinblastine; and Vincristine. Additional chemotherapy drugs are envisioned, and are listed in chemocare.com (http://www.chemocare.com/bio/default.sps).

In one embodiment of the instant articles of manufacture, the agent is soluble RAGE. In another embodiment, the agent is soluble RAGE G82S. In a further embodiment, the agent is an antibody directed to RAGE. In yet a further embodiment, the agent is an antibody directed to RAGE G82S.

This invention further provides an article of manufacture comprising a packaging material having therein an agent that inhibits binding between RAGE and/or RAGE G82S and a ligand thereof, wherein the packaging material has affixed thereto a label indicating a use for the agent for treating a glomerular injury in a subject. Finally, this invention provides an article of manufacture comprising a packaging material having therein an agent that inhibits binding between RAGE and/or RAGE G82S and a ligand thereof, wherein the packaging material has affixed thereto a label indicating a use for the agent for treating glomerulosclerosis, proteinuria or albuminuria in a subject.

In the preferred embodiment of the instant articles of manufacture, the subject is human. In one embodiment, the subject is not afflicted with diabetes. In another embodiment, the subject is receiving or is about to receive a chemotherapy drug. In a further embodiment, the chemotherapy drug is adriamycin. In yet a further embodiment, the chemotherapy drug is selected from the following: 5-fluorouracil; Actinomycin D; Alpha interferon; Bleomycin; Cisplatin; Cyclophosphamide; Dexamethasone; Doxorubicin; Epoetin alfa; Etoposide; Gleevec; Herceptin; Interferon alfa; Interleukin-2; Interleukin-11; Methotrexate; Neupogen; Nitrogen Mustard; Paclitaxel; Prednisolone; Prednisone; PROCRIT; Rituximab; Tamoxifen; Thalidomide; Vinblastine; and Vincristine. Additional chemotherapy drugs are envisioned, and are listed in chemocare.com (http://www.chemocare.com/bio/default.sps).

In one embodiment of the instant articles of manufacture, the agent is soluble RAGE. In another embodiment, the agent is soluble RAGE G82S. In a further embodiment, the agent is an antibody directed to RAGE. In yet a further embodiment, the agent is an antibody directed to RAGE G82S.

This invention is illustrated in the Experimental Details section which follows. This section is set forth to aid in an understanding of the invention but is not intended to, and should not be construed to, limit in any way the invention set forth in the claims which follow.

### EXPERIMENTAL DETAILS

Methods

Animal Studies

Male BALB/c mice at the age of six weeks received one intravenous dose of adriamycin (ADR), 10.5 mg/kg. Immediately after injection of ADR, mice received once daily administration of murine soluble RAGE, the extracellular ligand binding domain of RAGE, 100 μg per day, beginning immediately at the time of ADR treatment, and continued until the day of sacrifice.

Morphologic Studies

Dissected kidneys were fixed in buffered formalin (10%) overnight and then routinely processed for light microscopy. Fixed paraffin-embedded tissues were cut (3 μm thick) and mounted on slides coated with 3-aminopropyltriethoxy silane (Sigma) followed by incubation at 37° C. overnight. Light microscopic views after staining with periodic acid Schiff (PAS) were scanned into a computer and the quantification of areas of mesangial matrix and glomerulus was performed using a Zeiss microscope and image analysis system (MediaCybernetics). To calculate mesangial area, only nuclei-free regions were included. Forty glomeruli from each animal were selected at random on the stained sections (20 from the outer region and 20 from the inner region). Morphometry was performed by investigators blinded to the experimental protocol.

Functional Studies

Twenty-four hour urine collection was obtained from each animal using metabolic cages. Urine albumin and creatinine were determined using Albuwell M and creatinine assays from Exocell (Philadelphia, Pa.) according to the manufacturer's instructions.

Statistical Analysis

The mean±standard error (SE) of the mean is reported. Statistical significance (defined as p<0.05) was determined by ANOVA. Where indicated, post-hoc analysis was employed using Dunnett's t-test using StatView 4.0 (Abacus Concepts, Inc., Berkeley, Calif.).

Results

RAGE and Cellular Activation

It was in the context of roles for inflammatory cells and podocytes in the pathogenesis of FSGS that a role for Receptor for AGE (RAGE) was first speculated. RAGE is a multiligand member of the immunoglobulin superfamily of cell surface molecules (26-27) that engages distinct molecules; ligand-RAGE interaction activates cell signalling pathways (such as NF-kB; p44/p42, p38 and SAPK/JNK MAP kinases; cdc42/rac; and JAK/STAT, for example) (28-33) that are required for RAGE-mediated effects. Importantly, deletion of the cytosolic tail of RAGE imparts a dominant negative effect in cultured cells and in vivo.

RAGE is Principally Expressed in the Podocyte in the Glomerulus

The findings have demonstrated that the principal site of RAGE expression in the glomerulus is the podocyte, at low levels in homeostasis (34); podocyte RAGE expression is upregulated in human and murine diabetes (34).

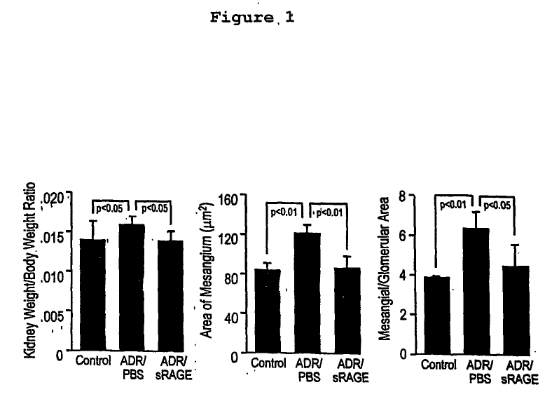

To address the concept that RAGE may be involved in the pathogenesis of ADR-mediated FSGS, a single injection of ADR, 10.5 mg/kg, to male BALB/c mice at age 6 weeks was administered. ADR-treated mice received once daily administration of murine soluble RAGE, the extracellular ligand binding domain of RAGE, 100 μg per day, beginning immediately at the time of ADR treatment, and continued until the day of sacrifice. Other ADR-treated mice received vehicle, PBS. At 2 and 6 weeks after ADR, kidney weight/body weight ratios were significantly decreased in sRAGE-treated vs. PBS-treated mice. Examination of mesangial area at 2 and 6 weeks after ADR revealed that in a time-dependent manner, ADR administration was associated with increased mesangial area, and increased mesangial matrix/glomerular area fraction by PAS staining (FIGS. 1&2, respectively). At 2 and 6 weeks, administration of sRAGE resulted in significantly decreased mesangial area and mesangial/glomerular area compared with PBS treatment (FIGS. 1&2, respectively).

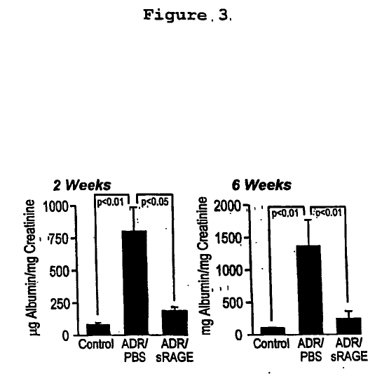

The key test of these concepts was the degree to which blockade of RAGE would suppress the development of albuminuria. Mice were placed in metabolic cages and 24 hr urine collected. Urine levels of albumin and creatinine were determined; results are reported as μg albumin/μg creatinine. At 2 weeks after ADR, PBS-treated mice displayed an ≈10-fold increase in urine albumin/creatinine compared to saline-treated mice not receiving ADR (809.55±365.85 vs. 85.78±17.56 albumin/creatinine; p<0.01) (FIG. 3). In mice receiving ADR and sRAGE, levels of albumin/creatinine were markedly reduced (191.08±49.93; p<0.05 vs. PBS-treated mice receiving ADR) (FIG. 3). At six weeks, the results were similarly striking. PBS-treated mice receiving ADR displayed urine albumin/creatinine of 1,362.96±987.97 vs 84.47±49.93 in control mice not receiving ADR; p<0.01 (FIG. 3). In the presence of sRAGE, ADR-mediated albuminuria was significantly reduced, to 249.76±283.19 μg albumin/creatinine; p<0.01 vs PBS/ADR (FIG. 3).

Taken together, these findings strongly support the hypothesis that RAGE activation importantly contributes to mechanisms linked to glomerular injury. Administration of soluble RAGE afforded significant protection against the morphologic and functional indices of glomerular injury upon administration of glomerulosclerosis-inducing agents. RAGE blockade is proposed as a new means to prevent glomerular injury in this class of diseases.

### REFERENCES

1. Bolton, W. K., and Abdel-Rahman, E. Pathogenesis of focal glomerulosclerosis. Nephron 88:6-13, 2001.
2. Bertolatus, J. A., and Hunsicker, L. G. Glomerular sieving of anionic and neutral bovine albumins in proteinuric rats. Kidney International 28:467-476, 1985.
3. Whiteside, C., Prutis, K., Cameron, R., and Thompson, J. Glomerular epithelial detachment, not reduced charge density, correlates with proteinuria in adriamycin and puromycin nephrosis. Lab Investigation 61:650-660, 1989.
4. Weening, J. J., and Rennke, H. G. Glomerular permeability and polyanion in adriamycin nephrosis in the rat. Kidney International 24:152-159, 1983.
5. Salant, D. J., Belok, S., Stilmant, M. M., Darby, C., and Couser, W. G. Determinants of glomerular localization of subepithelial immune deposits: effects of altered antigen to antibody ratio, steroids, vasoactive amine antagonists, and aminonucleoside of puromycin on passive Heymann nephritis in rats. Lab. Investigation 41:89-99, 1979.
6. Chen, A., Ding, S. L, Sheu, L. F., Song, Y. B., Shich, S. D., Shaio, M. F., Chou, W. Y., and Ho, Y. S. Experimental IgA nephropathy: enhanced deposition of glomerular IgA immune complex in proteinuric states. Lab Investigation 70:639-647, 1995.
7. Chen, A., Wei, C. H., Sheu, L. F., Ding, S. L., and Lee, W. H. Induction of proteinuria by adriamycin or bovine serum albumin in the mouse. Nephron 69:293-300, 1995.
8. Wang, Y., Wang, Y. P., Tay, Y. C., and Harris, D. C. H. Progressive adriamycin nephropathy in mice: sequence of histologic and immunohistochemical events. Kidney International 58:1797-1804, 2000.
9. Wang, Y., Wang, Y. P., Tay, Y. C., and Harris, D. C. H. Role of CD8+ cells in the progression of murine adriamycin nephropathy. Kidney International 59:941-949, 2001.
10. Bariety, J., Nochy, D., Mandet, C., Jacquot, C., Glotz, D., and Meyrier, A. Podocytes undergo phenotypic changes and express macrophage-associated markers in idiopathic collapsing glomerulopathy. Kidney International 53:918-925, 1998.
11. Schwartz, M. M., Evans, J., Bain, R. P., Korbet, S. M. Focal segmental glomerulosclerosis: prognostic implications of the cellular lesion (abstract). J. American Society of Nephrology 10: 1900-1907, 1999.
12. Magil, A., and Cohen, A. H. Monocytes and focal glomerulosclerosis. Lab Investigation 61:404-409, 1989.
13. Fries, J. W., Sandstrom, D. J., Meyer, T. W., and Rennke, H. G. Glomerular hypertrophy and epithelial cell injury modulate progressive glomerulosclerosis in the rat. Lab. Investigation 60:205-218, 1989.
14. Kriz, W., Gretz, N., and Lemley, K. V. Progression of glomerular diseases: is the podocyte the culprit? Kidney International 54:687-697, 1998.
15. Kerjaschki, D. Dysfunctions of cell biological mechanisms of visceral epithelial cell (podocytes) in glomerular diseases. Kidney International 45: 300-313, 1994.
16. Schiffer, M., Bitzer, M., Roberts, I. S. D., Kopp, J. B., ten Dijke, P., Mundel, P., and Bottinger, E. P. Apoptosis in podocytes induced by TGF-β and Smad7. J. Clinical Investigation 108:807:816, 2001.
17. Sharma K., Jin Y., Guo J., Ziyadeh F. N. Neutralization of TGF-β by anti-TGF-β antibody attenuates kidney hypertrophy and the enhanced extracellular matrix gene expression in stz-induced diabetic mice. Diabetes 45:522-530, 1996.
18. Ziyadeh F. N., Hoffman B. B., Han D. C., Iglesias-De La Cruz M. C., Hong S. W., Isono M., Chen S., McGowan T. A., and Sharma K. Long-term prevention of renal insufficiency, excess matrix gene expression, and glomerular mesangial matrix expansion by treatment with monoclonal antitransforming growth factor-β antibody in db/db diabetic mice. Proc. Natl. Acad. Sci. USA. 97:8015-8020, 2000.
19. Koya, D., Haneda, M., Nakagawa, H., Takagi, C., Xia, P., Clermont, A., Bursell, S. E., Kern, T. S., Ballas, L. M., Heath, W. F., Stramm, L. E., Feener, E. P., and King, G. L. Amelioration of accelerated diabetic mesangial expansion by treatment with a PKC beta inhibitor in diabetic db/db mice, a rodent model of type 2 diabetes. FASEB J 14:439-447, 2000.
20. Pavenstadt H. Roles of the podocyte in glomerular function. Am J Physiol Renal Physiol 278:F173-F179, 2000.
21. Nakamura T., Ushiyama C., Osada S., Hara M., Shimada N., and Koide H. Pioglitazone reduces urinary podocyte excretion in type 2 diabetes patients with microalbuminuria. Metabolism 50:1193-1196, 2000.
22. Hoshi S., Shu Y., Yoshida F., Inagaki T., Sonoda J., Watanable T., Nomoto K., and Nagata M. Podocyte injury promotes progressive nephropathy in Zucker diabetic fatty rats. Lab. Invest. 82:25-35, 2002.
23. Mifsud S. A., Allen T. J., Bertram J. F., Hulthen U. L., Kelly D. J., Cooper M. E., Wilkinson-Berka J. L., and Gilbert R. E. Podocyte foot process broadening in experimental diabetic nephropathy: amelioration with renin-angiotensin blockade. Diabetologia 44:878-882, 2001.
24. Cooper M. E., Vranes D., Youssef S., Stacker S. A., Cox A. J., Rizkalla B., Casley D. J., Bach L. A., Kelly D. J., and Gilbert R. E. Increased renal expression of vascular endothelial growth factor (VEGF) and its receptor VEGFR-2 in experimental diabetes. Diabetes 48:2229-2239, 1999.
25. De Vriese A., Tilton R., and Vanholder R. Hyperfiltration and albuminuria in diabetes: role of vascular endothelial growth factor (VEGF) (Abstract) J. American Soc. Nephrol. 10:A3434, 1999.
26. Schmidt, A. M., Vianna, M., Gerlach, M., Brett, J., Ryan, J., Kao, J., Esposito, C., Hegarty, H., Hurley, W., Clauss, M., Wang, F., Pan, Y. C., Tsang, T. C., and Stern, D. Isolation and characterization of binding proteins for advanced glycosylation endproducts from lung tissue which are present on the endothelial cell surface. J. Biol. Chem. 267:14987-14997, 192.
27. Neeper, M., Schmidt, A. M., Brett, J., Yan, S. D., Wang, F., Pan, Y. C., Elliston, K., Stern, D., and Shaw, A. Cloning and expression of RAGE: a cell surface receptor for advanced glycosylation end products of proteins. J. Biol. Chem. 267:14998-15004, 1992.
28. Yan, S-D., Schmidt A-M., Anderson, G., Zhang, J., Brett, J., Zou, Y-S., Pinsky, D., and Stern, D. Enhanced cellular oxidant stress by the interaction of advanced glycation endproducts with their receptors/binding proteins. J. Biol. Chem. 269:9889-9897, 1994.
29. Lander, H. L., Tauras, J. M., Ogiste, J. S., Moss, R. A., and A. M. Schmidt. Activation of the Receptor for Advanced Glycation Endproducts triggers a MAP Kinase pathway regulated by oxidant stress. J. Biol. Chem. 272:17810-17814, 1997.
30. Taguchi, A., Blood, D. C., del Toro, G., Canet, A., Lee, D. C., Qu, W., Tanji, N., Lu, Y, Lalla, E., Fu, C., Hofmann, M. A., Kislingler, T., Ingram, M., Lu, A., Tanaka, H., Hori, O., Ogawa, S., Stern, D. M., and Schmidt, A. M. Blockade of amphoterin/RAGE signalling suppresses tumor growth and metastases. Nature 405:354-360, 2000.
31. Hofmann, M. A., Drury, S., Fu, C., Qu, W., Taguchi, A., Lu, Y., Avila, C., Kambham, N., Bierhaus, A., Nawroth, P., Neurath, M. F., Slattery, T., Beach, D., McClary, J., Nagashima, M., Morser, J., Stern, D., and Schmidt, A. M. RAGE mediates a novel proinflammatory axis: a central cell surface receptor for S100/calgranulin polypeptides. Cell 97:889-901, 1999.
32. Huttunen H. J., Fages C., Rauvala H. RAGE-mediated neurite outgrowth and activation of NF-kB require the cytosolic domain of the receptor but different downstream signalling pathways. Journal of Biological Chemistry 274:19919-19924, 1999.
33. Kislinger, T., Fu, C., Huber, B., Qu, W., Taguchi, A., Yan, S. D., Hofmann, M., Yan, S. F., Pischetsrider, M., Stern, D., and Schmidt, A. M. N^{e}(carboxymethyl)lysine modifications of proteins are ligands for RAGE that activate cell signaling pathways and modulate gene expression. J. Biol. Chemistry 274: 31740-31749, 1999.
34. Tanji, N., Markowitz, G. S., Fu, C., Kislinger, T., Taguchi, A., Pischetsrieder, M., Stern, D., Schmidt, A. M., and D'Agati, V. D. The expression of Advanced, Glycation Endproducts and their cellular receptor RAGE in diabetic nephropathy and non-diabetic renal disease. J. American Soc. Nephrol. 11:1656-1666, 2000.
35. U.S. Ser. No. 08/755,235, filed Nov. 22, 1996, Publication No. US-2003-0059423-A1, published Mar. 27, 2003.

1. A method for inhibiting the onset of a glomerular injury in a subject comprising administering to the subject a prophylactically effective amount of an agent that inhibits binding between RAGE and/or RAGE G82S and a ligand thereof.

2-15. (canceled)

16. A method for treating a glomerular injury in a subject comprising administering to the subject a therapeutically effective amount of an agent that inhibits binding between RAGE and/or RAGE G82S and a ligand thereof.

17-28. (canceled)

29. A method for inhibiting the onset of glomerulosclerosis, proteinuria or albunuria in a subject comprising administering to the subject a prophylactically effective amount of an agent that inhibits binding between RAGE and/or RAGE G82S and a ligand thereof.

30-39. (canceled)

40. A method for treating glomerulosclerosis, proteinuria or albunuria in a subject comprising administering to the subject a therapeutically effective amount of an agent that inhibits binding between RAGE and/or RAGE G82S and a ligand thereof.

41-70. (canceled)
